SEQUENCE LISTING (14 sequences)

SEQ ID NO: 1 - DNA, 1989 bp, Citrullus lanatus
atggcggcggctgcttccccatgcgcctccaaatccttctccaaaccctcctccttctcccttcctaaacccttctccagatttgatctttcattttcccctcttcctcacaatccctccttccgccgtcctcttcgcatctgcagctcctctctctccaatccttcccccaaaccctcttccaccgccgcctccgccgctgccgtcgacacttccaccatcgccagccctgagattttcgtctcccgatttgccgctgatgaaccgagaaaaggcgccgatatcctcgtcgaggctctcgagcgccaaggcgtcaccaatgtcttcgcttaccccggcggcgcgtccatggagatccatcaggccctcacccgctcttcaattatccgtaacgttctccctcgccacgagcagggcggcgtattcgccgccgagggttacgctcgctcctctggccttcccggcgtttgcattgcaacttccggccccggtgccactaatctggtcagcggccttgccgacgccctcctcgacagcgttccattggtggccattacagggcaagttccgagaagaatgatcggtaccgatgcgtttcaggaaactcccattgttgaggtaactcgctctattacaaagcacaactatctggttcttgatgtagatgacattcctaggattgtgagcgaggcatttttcctcgcaatttctggtcgacctggtcctgttcttattgatattcctaaggacgttcagcaacagctcgcggttcccaattggaatcagcccatgaaattacctggttacttgtctaggttacctaagcctccaattgactctcatttggaacaaattgtgcgattggtatctgaatctaagaagcctgttctttacgtgggtggagggtgtttgaattcctcggaggagctgcggcgttttgtcaagctcaccgggattccggtggccagcactctaatgggtctcggggcttatccttgctccggtgaattgtctctgcaaatgctgggaatgcatgggactgtctatgccaactatgctgtggataaatctgatctgttgcttgcatttggagtgagatttgatgaccgtgtgacagggaagctcgaggcctttgcaagccgtgcaaaaatcgttcatatcgatattgattcggctgagattggaaagaacaagcagccccatgtgtcggtctgtggtgatgtaaagttggccttacagggaatgaatcgtttacttgagaaaactgagaagcttcactttgatttctctgcgtggagagaggagctgaatgagcagaaatcaaagtatccattgactttcaaaacatttgatgaggccattcctccacagtatgcaattcaacttcttgatgagctaaccaacggcgaggccatcgtaagcacaggggtggggcagcatcagatgtgggcagctcagttctacaagtacaagaagccacgtcagtggttaacctctggagggttaggtgcaatgggctttggactgccagcagccatgggggcagcagtagcaaatcccgatgcggtggtcgtagacatcgacggggacgggagtttcatcatgaatgtccaagaactggcaacaatcagtgttgagaaacttccagtgaagatcctgctgctgaacaaccagcatctaggcatggttgtgcagtgggaggatcggttctataaagccaacagagctcacacttacctgggagatccatcaaacgagaccgagatattccccaacatgttgaagttcgcagaggcctgcgggatcccagctgcccgtgtgacgaagagagctgagttaagagcagcaatgaagaagatgctggaaacagaagggccttacttgttggatgtgattgtaccgcatcaggaacatgtcctgccgatgattcccagtggcggtgctttcaaagatgtgatcacagagggagatggaagatccgtatactaa

SEQ ID NO: 2 - protein, 662 aa, Citrullus lanatus
MAAAASPCASKSFSKPSSFSLPKPFSRFDLSFSPLPHNPSFRRPLRICSSSLSNPSPKPSSTAASAAAVDTSTIASPEIFVSRFAADEPRKGADILVEALERQGVTNVFAYPGGASMEIHQALTRSSIIRNVLPRHEQGGVFAAEGYARSSGLPGVCIATSGPGATNLVSGLADALLDSVPLVAITGQVPRRMIGTDAFQETPIVEVTRSITKHNYLVLDVDDIPRIVSEAFFLAISGRPGPVLIDIPKDVQQQLAVPNWNQPMKLPGYLSRLPKPPIDSHLEQIVRLVSESKKPVLYVGGGCLNSSEELRRFVKLTGIPVASTLMGLGAYPCSGELSLQMLGMHGTVYANYAVDKSDLLLAFGVRFDDRVTGKLEAFASRAKIVHIDIDSAEIGKNKQPHVSVCGDVKLALQGMNRLLEKTEKLHFDFSAWREELNEQKSKYPLTFKTFDEAIPPQYAIQLLDELTNGEAIVSTGVGQHQMWAAQFYKYKKPRQWLTSGGLGAMGFGLPAAMGAAVANPDAVVVDIDGDGSFIMNVQELATISVEKLPVKILLLNNQHLGMVVQWEDRFYKANRAHTYLGDPSNETEIFPNMLKFAEACGIPAARVTKRAELRAAMKKMLETEGPYLLDVIVPHQEHVLPMIPSGGAFKDVITEGDGRSVY

SEQ ID NO: 3 - DNA, 1989 bp, Citrullus lanatus
atggcggcggctgcttccccatgcgcctccaaatccttctccaaaccctcctccttctcccttcctaaacccttctccagatttgatctttcattttcccctcttcctcacaatccctccttccgccgtcctcttcgcatctgcagctcctctctctccaatccttcccccaaaccctcttccaccgccgcctccgccgctgccgtcgacacttccaccatcgccagccctgagattttcgtctcccgatttgccgctgatgaaccgagaaaaggcgccgatatcctcgtcgaggctctcgagcgccaaggcgtcaccaatgtcttcgcttaccccggcggcgcgtccatggagatccatcaggccctcacccgctcttcaattatccgtaacgttctccctcgccacgagcagggcggcgtattcgccgccgagggttacgctcgctcctctggccttcccggcgtttgcattgcaacttccggccccggtgccactaatctggtcagcggccttgccgacgccctcctcgacagcgttccattggtggccattacagggcaagtttcgagaagaatgatcggtaccgatgcgtttcaggaaactcccattgttgaggtaactcgctctattacaaagcacaactatctggttcttgatgtagatgacattcctaggattgtgagcgaggcatttttcctcgcaatttctggtcgacctggtcctgttcttattgatattcctaaggacgttcagcaacagctcgcggttcccaattggaatcagcccatgaaattacctggttacttgtctaggttacctaagcctccaattgactctcatttggaacaaattgtgcgattggtatctgaatctaagaagcctgttctttacgtgggtggagggtgtttgaattcctcggaggagctgcggcgttttgtcaagctcaccgggattccggtggccagcactctaatgggtctcggggcttatccttgctccggtgaattgtctctgcaaatgctgggaatgcatgggactgtctatgccaactatgctgtggataaatctgatctgttgcttgcatttggagtgagatttgatgaccgtgtgacagggaagctcgaggcctttgcaagccgtgcaaaaatcgttcatatcgatattgattcggctgagattggaaagaacaagcagccccatgtgtcggtctgtggtgatgtaaagttggccttacagggaatgaatcgtttacttgagaaaactgagaagcttcactttgatttctctgcgtggagagaggagctgaatgagcagaaatcaaagtatccattgactttcaaaacatttgatgaggccattcctccacagtatgcaattcaacttcttgatgagctaaccaacggcgaggccatcgtaagcacaggggtggggcagcatcagatgtgggcagctcagttctacaagtacaagaagccacgtcagtggttaacctctggagggttaggtgcaatgggctttggactgccagcagccatgggggcagcagtagcaaatcccgatgcggtggtcgtagacatcgacggggacgggagtttcatcatgaatgtccaagaactggcaacaatcagtgttgagaaacttccagtgaagatcctgctgctgaacaaccagcatctaggcatggttgtgcagtgggaggatcggttctataaagccaacagagctcacacttacctgggagatccatcaaacgagaccgagatattccccaacatgttgaagttcgcagaggcctgcgggatcccagctgcccgtgtgacgaagagagctgagttaagagcagcaatgaagaagatgctggaaacagaagggccttacttgttggatgtgattgtaccgcatcaggaacatgtcctgccgatgattcccagtggcggtgctttcaaagatgtgatcacagagggagatggaagatccgtatactaa

SEQ ID NO: 4 - protein, 662 aa, Citrullus lanatus
MAAAASPCASKSFSKPSSFSLPKPFSRFDLSFSPLPHNPSFRRPLRICSSSLSNPSPKPSSTAASAAAVDTSTIASPEIFVSRFAADEPRKGADILVEALERQGVTNVFAYPGGASMEIHQALTRSSIIRNVLPRHEQGGVFAAEGYARSSGLPGVCIATSGPGATNLVSGLADALLDSVPLVAITGQVSRRMIGTDAFQETPIVEVTRSITKHNYLVLDVDDIPRIVSEAFFLAISGRPGPVLIDIPKDVQQQLAVPNWNQPMKLPGYLSRLPKPPIDSHLEQIVRLVSESKKPVLYVGGGCLNSSEELRRFVKLTGIPVASTLMGLGAYPCSGELSLQMLGMHGTVYANYAVDKSDLLLAFGVRFDDRVTGKLEAFASRAKIVHIDIDSAEIGKNKQPHVSVCGDVKLALQGMNRLLEKTEKLHFDFSAWREELNEQKSKYPLTFKTFDEAIPPQYAIQLLDELTNGEAIVSTGVGQHQMWAAQFYKYKKPRQWLTSGGLGAMGFGLPAAMGAAVANPDAVVVDIDGDGSFIMNVQELATISVEKLPVKILLLNNQHLGMVVQWEDRFYKANRAHTYLGDPSNETEIFPNMLKFAEACGIPAARVTKRAELRAAMKKMLETEGPYLLDVIVPHQEHVLPMIPSGGAFKDVITEGDGRSVY

SEQ ID NO: 5 - DNA, 1989 bp, Citrullus lanatus
atggcggcggctgcttccccatgcgcctccaaatccttctccaaaccctcctccttctcccttcctaaacccttctccagatttgatctttcattttcccctcttcctcacaatccctccttccgccgtcctcttcgcatctgcagctcctctctctccaatccttcccccaaaccctcttccaccgccgcctccgccgctgccgtcgacacttccaccatcgccagccctgagattttcgtctcccgatttgccgctgatgaaccgagaaaaggcgccgatatcctcgtcgaggctctcgagcgccaaggcgtcaccaatgtcttcgcttaccccggcggcgcgtccatggagatccatcaggccctcacccgctcttcaattatccgtaacgttctccctcgccacgagcagggcggcgtattcgccgccgagggttacgctcgctcctctggccttcccggcgtttgcattgcaacttccggccccggtgccactaatctggtcagcggccttgccgacgccctcctcgacagcgttccattggtggccattacagggcaagttttgagaagaatgatcggtaccgatgcgtttcaggaaactcccattgttgaggtaactcgctctattacaaagcacaactatctggttcttgatgtagatgacattcctaggattgtgagcgaggcatttttcctcgcaatttctggtcgacctggtcctgttcttattgatattcctaaggacgttcagcaacagctcgcggttcccaattggaatcagcccatgaaattacctggttacttgtctaggttacctaagcctccaattgactctcatttggaacaaattgtgcgattggtatctgaatctaagaagcctgttctttacgtgggtggagggtgtttgaattcctcggaggagctgcggcgttttgtcaagctcaccgggattccggtggccagcactctaatgggtctcggggcttatccttgctccggtgaattgtctctgcaaatgctgggaatgcatgggactgtctatgccaactatgctgtggataaatctgatctgttgcttgcatttggagtgagatttgatgaccgtgtgacagggaagctcgaggcctttgcaagccgtgcaaaaatcgttcatatcgatattgattcggctgagattggaaagaacaagcagccccatgtgtcggtctgtggtgatgtaaagttggccttacagggaatgaatcgtttacttgagaaaactgagaagcttcactttgatttctctgcgtggagagaggagctgaatgagcagaaatcaaagtatccattgactttcaaaacatttgatgaggccattcctccacagtatgcaattcaacttcttgatgagctaaccaacggcgaggccatcgtaagcacaggggtggggcagcatcagatgtgggcagctcagttctacaagtacaagaagccacgtcagtggttaacctctggagggttaggtgcaatgggctttggactgccagcagccatgggggcagcagtagcaaatcccgatgcggtggtcgtagacatcgacggggacgggagtttcatcatgaatgtccaagaactggcaacaatcagtgttgagaaacttccagtgaagatcctgctgctgaacaaccagcatctaggcatggttgtgcagtgggaggatcggttctataaagccaacagagctcacacttacctgggagatccatcaaacgagaccgagatattccccaacatgttgaagttcgcagaggcctgcgggatcccagctgcccgtgtgacgaagagagctgagttaagagcagcaatgaagaagatgctggaaacagaagggccttacttgttggatgtgattgtaccgcatcaggaacatgtcctgccgatgattcccagtggcggtgctttcaaagatgtgatcacagagggagatggaagatccgtatactaa

SEQ ID NO: 6 - protein, 662 aa, Citrullus lanatus
MAAAASPCASKSFSKPSSFSLPKPFSRFDLSFSPLPHNPSFRRPLRICSSSLSNPSPKPSSTAASAAAVDTSTIASPEIFVSRFAADEPRKGADILVEALERQGVTNVFAYPGGASMEIHQALTRSSIIRNVLPRHEQGGVFAAEGYARSSGLPGVCIATSGPGATNLVSGLADALLDSVPLVAITGQVLRRMIGTDAFQETPIVEVTRSITKHNYLVLDVDDIPRIVSEAFFLAISGRPGPVLIDIPKDVQQQLAVPNWNQPMKLPGYLSRLPKPPIDSHLEQIVRLVSESKKPVLYVGGGCLNSSEELRRFVKLTGIPVASTLMGLGAYPCSGELSLQMLGMHGTVYANYAVDKSDLLLAFGVRFDDRVTGKLEAFASRAKIVHIDIDSAEIGKNKQPHVSVCGDVKLALQGMNRLLEKTEKLHFDFSAWREELNEQKSKYPLTFKTFDEAIPPQYAIQLLDELTNGEAIVSTGVGQHQMWAAQFYKYKKPRQWLTSGGLGAMGFGLPAAMGAAVANPDAVVVDIDGDGSFIMNVQELATISVEKLPVKILLLNNQHLGMVVQWEDRFYKANRAHTYLGDPSNETEIFPNMLKFAEACGIPAARVTKRAELRAAMKKMLETEGPYLLDVIVPHQEHVLPMIPSGGAFKDVITEGDGRSVY

SEQ ID NO: 7 - DNA, 23 bp, Artificial sequence
  Synthesized
attgaagttccgagaagaatgat

SEQ ID NO: 8 - DNA, 23 bp, Artificial sequence
  Synthesized
aaacatcattcttctcggaactt

SEQ ID NO: 9 - DNA, 20 bp, Artificial sequence
  Synthesized
gaagttccgagaagaatgat

SEQ ID NO: 10 - DNA, 25 bp, Artificial sequence
  Synthesized
catacctcccagaacacaaataagc

SEQ ID NO: 11 - DNA, 25 bp, Artificial sequence
  Synthesized
actgaagggcaatagtgaagaatgt

SEQ ID NO: 12 - DNA, 21 bp, Artificial sequence
  Synthesized
cgtcaccaatgtcttcgctta

SEQ ID NO: 13 - DNA, 24 bp, Artificial sequence
  Synthesized
caggcttcttagattcagatacca

SEQ ID NO: 14 - DNA, 19 bp, Artificial sequence
  Synthesized
aagttccgagaagaatgat

(19) United States
(12) Patent Application Publication
(10) Pub. No.: US 2020/0340007 A1
(43) Pub. Date: Oct. 29, 2020

(54) CLALS PROTEIN, ITS CODING GENE AND USE IN PREDICTING THE HERBICIDE RESISTANCE OF WATERMELON

(71) Applicant: BEIJING ACADEMY OF AGRICULTURE AND FORESTRY SCIENCES, Beijing (CN)

(72) Inventors: Yong XU, Beijing (CN); Shouwei TIAN, Beijing (CN); Linjian JIANG, Beijing (CN); Haiying ZHANG, Beijing (CN); Guoyi GONG, Beijing (CN); Shaogui GUO, Beijing (CN); Jie ZHANG, Beijing (CN); Yi REN, Beijing (CN); Honghe SUN, Beijing (US); Maoying LI, Beijing (US)

(21) Appl. No.: 16/923,838

(22) Filed: Jul. 8, 2020

Related U.S. Application Data
(63) Continuation of application No. PCT/CN2019/087744, filed on May 21, 2019.

(30) Foreign Application Priority Data
May 23, 2018 (CN) 201810500524.5

(51) Int. Cl. C12N 15/82

(52) U.S. Cl. CPC C12N 15/8278; C12Y 202/01006

(57) ABSTRACT

The present invention discloses a CLALS protein, its coding gene and use in predicting the herbicide resistance of a watermelon. The herbicide resistance of a watermelon to be tested in which “the amino acid residue at position 190 from N-terminus of the CLALS protein is only a non-proline residue” or a watermelon to be tested in which “the amino acid residue at position 190 from N-terminus of the CLALS protein is a non-proline residue and a proline residue” is stronger than that of a watermelon to be tested in which “the amino acid residue at position 190 from N-terminus of the CLALS protein is only a proline residue”. Experiments have shown that the type of the amino acid residue at position 190 from the N-terminus of CLALS protein can be used as a detection target to predict the herbicide resistance of a watermelon to be tested. The invention has great application value.

### RELATED APPLICATIONS

The present application is a continuing application of PCT Patent Application Number PCT/CN2019/087744, filed May 21, 2019, which claims priority to Chinese Application Number 201810500524.5, filed May 23, 2018, each of which is incorporated herein by reference in its entirety.

### TECHNICAL FIELD

The present invention belongs to the field of biotechnology, and particularly relates to a CLALS proteins, its coding genes, and use in predicting of the herbicide resistance of watermelon, variety improvement, and especially in culturing herbicide resistant traits.

### BACKGROUND OF THE INVENTION

Weed in watermelon field is one of the important factors limiting watermelon yield, quality and cost efficiency. Due to the low planting density of watermelon, the area of the bare ground in the field is large before the stems are spread. In addition, since the growing environment of watermelon is hot and humid, weeds are prone to occur. Therefore, compared with other crops, weed harmfulness is particularly significant in watermelon field. Artificial weeding in watermelon field is laborious and laborious. Chemical herbicide often causes medicinal harm to watermelons due to improper selection and doses, resulting in reduced yield and quality of watermelons. Weed makes the watermelon production to be reduced by about 20%, accounting for more than 30% of field labor costs. Therefore, weeding costs and herbicide safety issues in watermelon field have become restrictive factors affecting the development of the watermelon industry. Culturing watermelon varieties with the herbicide resistance can not only solve the problem of weed harmfulness that affects watermelon production, but also meet the simplification requirements for production of watermelon, in which 1-2 herbicides are used to kill weed during planting, but it will not affect the growth of watermelon itself. Acetolactate synthase (ALS) is a key enzyme in the synthesis of branched chain amino acids in plants and microorganisms. ALS inhibitor herbicides inhibit the activity of ALS in plants, thereby preventing the synthesis of branched chain amino acids, which in turn affects protein synthesis and plant growth, and eventually causes plant death. ALS inhibitor herbicides have the advantages of high activity, strong selectivity, broad herbicidal spectrum, and low toxicity, and have become the most active class of commercial herbicides in 1990s.

In April 2016, Komor et al. adopted a method of fusing Cas9 variant, cytidine deaminase (CD) and uracil DNA glycosylase inhibitor (UGI), and achieved efficient single base site-directed mutations in rats. Based on the same principle, an efficient plant single base editing system nCas9-PBE has been constructed by fusing Cas9 variant (nCas9-D10A), rat cytosine deaminase (rAPOBEC1) and uracil glycosylase inhibitor (UGI), by which a single base site-directed mutation of target genes has been achieved in crops such as rice, wheat, corn, and arabidopsis. nCas9-PBE can substitute C of a target site DNA with T, and the C base deamination window covers the 7 nucleotides of the target sequence (positions 3-9 from the far end of the PAM). This technique neither requires double-strand break (DSB) generated at the target site nor requires the involvement of a donor DNA. It has the characteristics of simplicity, adaptability and efficiency. The successful establishment and application of the nCas9-PBE single base editing system provide a reliable solution for the efficient and large-scale creation of single base mutants, and provide an important technical support for crop genetic improvement and culturing of new varieties.

### SUMMARY OF THE INVENTION

The object of the present invention is to culture watermelon varieties with the herbicide resistance.

Firstly, the present invention protects a CLALS protein, which may be W1) or W2) as follows:

W1) segment I, segment II, and segment III may be included in this order from the N-terminus to the C-terminus;

the segment II may be an amino acid residue;

the segment I may be a1) or a2) or a3) as follows:
a1) a polypeptide with the amino acid sequence shown as positions 1 to 189 from the N-terminus in sequence 2 in the sequence listing;
a2) a polypeptide related to the herbicide resistance obtained by substituting the polypeptide shown in a1) with one or more amino acid residues;
a3) a polypeptide having an identity of 80% or more with the polypeptide shown in a1) or a2), derived from watermelon and related to the herbicide resistance;

the segment III may be b1) or b2) or b3) as follows:
b1) a polypeptide with the amino acid sequence shown as positions 191 to 662 in sequence 2 in sequence listing from the N-terminus;
b2) a polypeptide related to the herbicide resistance obtained by substituting the polypeptide shown in b1) with one or more amino acid residues;
b3) a polypeptide having an identity of 80% or more with the polypeptide shown in b1) or b2), derived from watermelon and related to the herbicide resistance,

W2) a fusion protein obtained by attaching a tag to the N-terminus or/and C-terminus of W1).

In the above a3), the term “identity” used refers to a sequence similarity to a natural amino acid sequence. “Identity” includes an amino acid sequence having an identity of 80%, or 85% or higher, or 90% or higher, or 95% or higher with the amino acid sequence from position 1 to 189 from the N-terminus shown in sequence 2 of the sequence listing of the present invention.

In the above b3), the term “identity” used refers to a sequence similarity to a natural amino acid sequence. “Identity” includes amino acid sequences having an identity of 80%, or 85% or higher, or 90% or higher, or 95% or higher with the amino acid sequence from position 191 to 662 from the N-terminus shown in sequence 2 of the sequence listing of the present invention.

In the CLALS protein, the segment II may be a proline residue or a non-proline residue. The non-proline residue may specifically be a serine residue or a leucine residue.

The CLALS protein may be composed of the segment I, the segment II, and the segment III in this order from the N-terminus to the C-terminus.

The CLALS protein may specifically be c1) or c2) or c3) or c4) or c5) as follows:

c1) a protein with the amino acid sequence shown in sequence 2 in the sequence listing;

c2) a protein with the amino acid sequence shown in sequence 4 in the sequence listing;

c3) a protein with the amino acid sequence shown in sequence 6 in the sequence listing;

c4) a protein related to the herbicide resistance obtained by substituting with one or more amino acid residues and/or deletions and/or additions in the segment I and/or the segment III of the protein shown in c1) or c2) or c3);

c5) a protein having an identity of 80% or more with the protein shown in c1) or c2) or c3) or c4), derived from watermelon and related to the herbicide resistance.

In the above c5), the term “identity” used refers to a sequence similarity to a natural amino acid sequence. “Identity” includes amino acid sequences having an identity of 80%, or 85% or higher, or 90% or higher, or 95% or higher with the amino acid sequences shown in sequence 2, sequence 4 or sequence 6 in the sequence listing of the present invention.

A nucleic acid molecule encoding the CLALS protein also falls into the protection scope of the present invention.

The nucleic acid molecule encoding the CLALS protein may be a DNA molecule shown in d1) or d2) or d3) or d4) or d5) as follows:

d1) a DNA molecule with a nucleotide sequence shown in sequence 1 in the sequence listing;

d2) a DNA molecule with a nucleotide sequence shown in sequence 3 in the sequence listing;

d3) a DNA molecule with a nucleotide sequence shown in sequence 5 in the sequence listing;

d4) a DNA molecule that has an identity of 75% or more with the nucleotide sequence defined in d1) or d2) or d3), and encodes the CLALS protein;

d5) a DNA molecule that hybridizes to the nucleotide sequence defined in d1) or d2) or d3) under a stringent condition and encodes the CLALS protein.

Wherein the nucleic acid molecule may be DNA, such as cDNA, genomic DNA, or recombinant DNA; and the nucleic acid molecule may also be RNA, such as mRNA or hnRNA, and the like.

In the above d4), the term “identity” used refers to a sequence similarity to a natural amino acid sequence. “Identity” includes a nucleotide sequence having an identity of 80%, or 85% or higher, or 90% or higher, or 95% or higher with the nucleotide sequence encoding the protein composed of the amino acid sequence shown in sequence 2 in the sequence listing of the present invention, or with the nucleotide sequence encoding the protein composed of the amino acid sequence shown in sequence 4 in the sequence listing of the present invention, or with the nucleotide sequence encoding the protein composed of the amino acid sequence shown in sequence 6 in the sequence listing of the present invention.

Sequence 1 in the sequence listing consists of 1989 nucleotides. The nucleotide of sequence 1 in the sequence listing encodes the amino acid sequence shown in sequence 2 in the sequence listing. Sequence 3 in the sequence listing consists of 1989 nucleotides. The nucleotide of sequence 3 in the sequence listing encodes the amino acid sequence shown in sequence 4 in the sequence listing. Sequence 5 in the sequence listing consists of 1989 nucleotides. The nucleotide of sequence 5 in the sequence listing encodes the amino acid sequence shown in sequence 6 in the sequence listing.

In the above, the identity may be evaluated with the naked eye or computer software. Using computer software, the identity between two or more sequences may be expressed as a percentage (%), which can be used to evaluate the identity between related sequences.

An expression cassette, a recombinant vector, a recombinant microorganism, or a transgenic cell line containing the nucleic acid molecule also falls into the protection scope of the present invention.

The invention also protects Z1) or Z2):

Z1) use of the CLALS protein or a nucleic acid molecule encoding the CLALS protein in regulating the herbicide resistance of watermelon;

Z2) use of the CLALS protein or a nucleic acid molecule encoding the CLALS protein in culturing watermelon with altered herbicide resistance.

The invention also protects a method for predicting the herbicide resistance of a watermelon to be tested.

As for the method for predicting the herbicide resistance of a watermelon to be tested protected by the present invention, it may specifically be S1): detecting the type of the amino acid residue at position 190 from the N-terminus of the CLALS protein of a watermelon to be tested. The herbicide resistance of a watermelon to be tested in which “the type of the amino acid residue at position 190 from the N-terminus of the CLALS protein is only a non-proline residue” or a watermelon to be tested in which “the type of the amino acid residue at position 190 from the N-terminus of the CLALS protein is a non-proline residue and a proline residue” is stronger than that of a watermelon to be tested in which “the type of the amino acid residue at position 190 from the N-terminus of the CLALS protein is only a proline residue”.

In the above S1), the amino acid type of the non-proline residue in “the type of the amino acid residue at position 190 from the N-terminus of the CLALS protein is only a non-proline residue” may be one or two.

As for the method for predicting the herbicide resistance of a watermelon to be tested protected by the present invention, it may specifically be S2): detecting the nucleotide sequence of the 190^{th} codon in the specific transcript of the total RNA of a watermelon to be tested. The specific transcript is the RNA transcribed from the gene encoding the CLALS protein, and the first codon of which is the start codon. The herbicide resistance of a watermelon to be tested in which “the nucleotide sequence of the 190^{th} codon in a specific transcript only encodes a non-proline” or a watermelon to be tested in which “the nucleotide sequence of the 190^{th} codon in a specific transcript only encodes a non-proline and a proline” is stronger than that of a watermelon to be tested in which “the nucleotide sequence of the 190^{th} codon in a specific transcript only encodes a proline”.

In the above S2), the amino acid type of the non-proline in “the nucleotide sequence of the 190^{th} codon in a specific transcript only encodes a non-proline” may be one or two.

As for the method for predicting the herbicide resistance of a watermelon to be tested protected by the present invention, it may specifically be S3): detecting the type of the nucleotide at positions 568 and 569 from 5′ end of the gene encoding the CLALS protein in the total DNA of a watermelon to be tested. The herbicide resistance of a watermelon to be tested in which “the type of the nucleotides at positions 568 and 569 from 5′ end of the gene encoding the CLALS protein is only c” is weaker than F1 or F2 or F3. F1 is a watermelon to be tested in which the type of the nucleotides at both positions 568 and 569 from 5′ end of the gene encoding the CLALS protein does not include c. F2 is a watermelon to be tested in which the type of the nucleotides at position 569 from 5′ end of the gene encoding the CLALS protein includes c, and the type of the nucleotides at position 568 does not include c. F3 is a watermelon to be tested in which the type of the nucleotides at position 568 from 5′ end of the gene encoding the CLALS protein includes c, and the type of the nucleotides at position 569 does not include c.

Specifically, the method for predicting the herbicide resistance of a watermelon to be tested protected by the present invention may include the following steps: detecting whether the total DNA of a watermelon to be tested has a DNA molecule shown in sequence 1 of the sequence listing, a DNA molecule shown in sequence 3 of the sequence listing, and a DNA molecule shown in sequence 5 of the sequence listing.

The herbicide resistance of a watermelon to be tested in which “the total DNA of the watermelon to be tested has a DNA molecule shown in sequence 3 of the sequence listing and/or a DNA molecule shown in sequence 5 of the sequence listing” is stronger than that of a watermelon to be tested in which “the total DNA of the watermelon to be tested just has a DNA molecule shown in sequence 1 of the sequence listing”.

The present invention also protects use of a substance A, a substance B or a substance C in predicting the herbicide resistance of a watermelon to be tested.

The substance A may be a substance for detecting the type of the amino acid residue at position 190 from the N-terminus of the CLALS protein.

The substance B may be a substance for detecting a nucleotide sequence of the 190^{th} codon in a specific transcript; the specific transcript is an RNA transcribed from a gene encoding the CLALS protein, and the first codon is the start codon.

The substance C may be a substance for detecting the type of nucleotide at positions 568 and 569 from the 5′ end of the gene encoding the CLALS protein.

The invention also protects use of a complete set of product A, a complete set of product B, or a complete set of product C in predicting the herbicide resistance of a watermelon to be tested.

The complete set of product A may be the substance A and a carrier recorded with a method A. The method A may be: the herbicide resistance of a watermelon to be tested in which “the type of the amino acid residue at position 190 from the N-terminus of the CLALS protein is only a non-proline residue” or a watermelon to be tested in which “the type of the amino acid residue at position 190 from the N-terminus of the CLALS protein is a non-proline residue and a proline residue” is stronger than that of a watermelon to be tested in which “the type of the amino acid residue at position 190 from the N-terminus of the CLALS protein is only a proline residue”.

The complete set of product B may be the substance B and a carrier recorded with a method B. The method B may be: the herbicide resistance of a watermelon to be tested in which “the nucleotide sequence of the 190^{th} codon in a specific transcript only encodes a non-proline” or a watermelon to be tested in which “the nucleotide sequence of the 190^{th} codon in a specific transcript encodes a non-proline and a proline” is stronger than that of a watermelon to be tested in which “the nucleotide sequence of the 190^{th} codon in a specific transcript only encodes a proline”.

The complete set of product C may be the substance C and a carrier recorded with a method C. The method C may be: the herbicide resistance of a watermelon to be tested in which “the type of the nucleotides at positions 568 and 569 from 5′ end of the gene encoding the CLALS protein is only c” is weaker than F1 or F2 or F3. F1 is a watermelon to be tested in which the type of the nucleotides at both positions 568 and 569 from 5′ end of the gene encoding the CLALS protein does not include c. F2 is a watermelon to be tested in which the type of the nucleotides at position 569 from 5′ end of the gene encoding the CLALS protein includes c, and the type of the nucleotides at position 568 does not include c. F3 is a watermelon to be tested in which the type of the nucleotides at position 568 from 5′ end of the gene encoding the CLALS protein includes c, and the type of the nucleotides at position 569 does not include c.

In the above use, the amino acid type of the non-proline residue in “the type of the amino acid residue at position 190 from the N-terminus of the CLALS protein is only a non-proline residue” may be one or two.

In the above use, the amino acid type of the non-proline in “the nucleotide sequence of the 190^{th} codon in a specific transcript only encodes a non-proline” may be one or two.

The invention also protects B1) or B2) or B3).

B1) use of the type of the amino acid residue at position 190 from the N-terminus of the CLALS protein as a detection target in predicting the herbicide resistance of a watermelon to be tested.

B2) use of the nucleotide sequence of the 190^{th} codon in a specific transcript as a detection target in predicting the herbicide resistance of a watermelon to be tested. The first codon is the start codon.

B3) use of the type of nucleotide at positions 568 and 569 from the 5′ end of the gene encoding the CLALS protein as a detection target in predicting the herbicide resistance of a watermelon to be tested.

Any of herbicides above may be one that targets the CLALS protein (i.e., an ALS inhibitor herbicide).

Any of ALS inhibitor herbicides above may be Y1) or Y2) or Y3) or Y4) or Y5): Y1) sulfonylurea herbicides; Y2) triazopyrimidine herbicides; Y3) triazolinone herbicides; Y4) pyrimidine salicylic acid herbicides; Y5) imidazolinones.

The sulfonylurea herbicides may be tribenuron, halosulfuron, bensulfuron, pyrazosulfuron, nicosulfuron, mesosulfuron, thiensulfuron, or rimsulfuron.

The triazopyrimidine herbicides can be specifically flumetsulam, penoxsulam, pyroxsulam, or florasulam.

The triazolinone herbicide may be flucarbazone.

The pyrimidinesalicylic acid herbicide may be bispyribac.

The imidazolinones may be Imazapic.

Any of the non-prolines mentioned above may be specifically serine or leucine.

A double-site or multiple-site mutant gene formed by a mutation of the amino acid residue at position 190 from the N-terminus of the CLALS protein and a mutation of other amino acid residues in the CLALS protein also falls into the protection scope of the present invention.

Use of the double-site or multiple-site mutant gene formed by a mutation of the amino acid residue at position 190 from the N-terminus of the CLALS protein and a mutation of other amino acid residues in the CLALS protein in regulating the herbicide resistance of watermelon also falls into the protection scope of the present invention.

In an embodiment of the present invention, a P190L mutant heterozygote and a P190S mutant heterozygote, and further a P190L homozygous mutant strain (type of the amino acid residue at position 190 from the N-terminus of the CLALS protein is only a leucine residue), a P190S homozygous mutant strain (type of the amino acid residue at position 190 from the N-terminus of the CLALS protein is only a serine residue), a P190L homozygous mutant strain (type of the amino acid residue at position 190 from the N-terminus of the CLALS protein are proline and leucine residues) and a P190S homozygous mutant (type of the amino acid residue at position 190 from the N-terminus of the CLALS protein are proline and serine residues) is obtained by the inventors through the plant single base editing system nCas9-PBE. Seedlings of the above mutants and seedlings of non-transgenic watermelon (the amino acid residue of the position 190 from the N-terminus of the CLALS protein is only a proline residue) were sprayed with tribenuron, and the results showed that the seedlings of non-transgenic watermelon die quickly (3-7 days after spraying tribenuron), the seedlings of the P190L heterozygous mutant strain, the seedlings of the P190S heterozygous mutant strain, the seedlings of the P190L homozygous mutant strain, and the seedlings of the P190S homozygous mutant strain all grew normally. Moreover, the seedlings of the P190L homozygous mutant strain and the seedlings of the P190S homozygous mutant strain have a better growth status than the seedlings of the P190L heterozygous mutant strain and the seedlings of the P190S heterozygous mutant strain.

Experiments have shown that the type of the amino acid residue at position 190 from the N-terminus of the CLALS protein can be used as a detection target, to predict the herbicide resistance of a watermelon to be tested. The invention has great application value.

### BRIEF DESCRIPTION OF THE DRAWINGS

FIG. 1 shows the identification results of the herbicide resistance.

FIGS. 2A-2G show the identification results of the herbicide resistance spectrum. FIG. 2A represents the control, FIG. 2B represents the watermelon seedlings sprayed with tribenuron, FIG. 2C represents the watermelon seedlings sprayed with halosulfuron, and FIG. 2D represents the watermelon seedlings sprayed with bensulfuron, FIG. 2E represents watermelon seedlings sprayed with pyrazosulfuron, FIG. 2F represents watermelon seedlings sprayed with flucarbazone, and FIG. 2G represents watermelon seedlings sprayed with flumetsulam.

### BEST MODE OF IMPLEMENTING THE INVENTION

The following examples are provided for better understanding of the present invention, but the present invention is not limited thereto. Unless otherwise specified, the experimental methods in the following examples are conventional methods. Unless otherwise specified, the test materials used in the following examples were purchased from conventional biochemical reagent stores. The quantitative experiments in the following examples all carried out three times, and the results were averaged.

The pBSE901 plasmid is described in the following literatures: Chen Y, Wang Z, Ni H, et al. CRISPR/Cas9-mediated base-editing system efficiently generates gain-of-function mutations in Arabidopsis [J]. Science China Life Sciences, 2017, 60 (5): 520-523.

BM culture medium: 0.44 g of MS culture medium, 3 g of sucrose and 0.8 g of agar were dissolved in 100 mL of deionized water, the pH value was adjusted to 5.8, and autoclaving was performed for 15 minutes. MS medium is a product of PhytoTech.

Co-culture medium: BM medium containing 1.5 mg/L of 6-BA.

Selective medium 1: a co-culture medium containing 100 mg/L of Timentin and 1.5 mg/L of Basta.

Selective medium 2: a co-culture medium containing 100 mg/L of Timentin and 2.0 mg/L of Basta.

Bud elongation medium: BM medium containing 0.1 mg/L of 6-BA, 0.01 mg/L of NAA, 100 mg/L of Timentin, and 1.5 mg/L of Basta.

Rooting medium: BM medium containing 1 mg/L of IBA.

### Example 1. Obtaining and Verifying Herbicide-Resistant Watermelon Mutants

The amino acid sequence of the CLALS protein is shown as sequence 2 in the sequence listing. The gene encoding the CLALS protein (i.e., the CLALS gene) is shown as sequence 1 in the sequence listing. The target sequence is selected based on the nucleotide sequence of the CLALS gene, and the target sequence has a nucleotide sequence of 5′-AAGTTCCGAGAAGAATGAT-3′ (sequence 14 in the sequence listing).

I. Construction of the Recombinant Plasmid pBSE901-ALS

1. pBSE901 plasmid is digested with restriction enzyme Bsa I, and a vector framework of about 15 Kb is recovered.

2. Primer ALS-190F: 5′-ATTGAAGTTCCGAGAAGAATGAT-3′ (sequence 7 in the sequence listing) (target sequence is shown as double underlined) and primer ALS-190R: 5′-AAACATCATTCTTCTCGGAACTT-3′ (sequence 8 in the sequence listing) (reverse complementary sequence of target sequence is shown as double underlined) are synthesized; and primer ALS-190F and primer ALS-190R are diluted with deionized water to 100 μM, respectively, to obtain a primer ALS-190F diluted solution and a primer ALS-190R diluted solution; and then annealing reaction is performed to form a DNA molecule I.

Annealing procedure: 95° C. water bath for 10 min, and naturally cooled to room temperature.

3. The vector framework is ligated to DNA molecule I, to obtain the recombinant plasmid pBSE901-ALS.

The recombinant plasmid pBSE901-ALS was sequenced. Based on the sequencing results, the structure of the recombinant plasmid pBSE901-ALS was described as follows: DNA molecule II is inserted into the restriction enzyme BsaI recognition sequence of pBSE901 plasmid. The DNA molecule II is 5′-GAAGTTCCGAGAAGAATGAT-3′ (sequence 9 in the sequence listing).

II. Preparation of Agrobacterium Infection Solution

1. The recombinant plasmid pBSE901-ALS constructed in step I is used to transform Agrobacterium tumefaciens EHA105 competent cells to obtain a recombinant Agrobacterium, named EHA105-pBSE901-ALS.

2. EHA105-pBSE901-ALS monoclone was inoculated into 20 ml of YEB liquid medium containing 50 mg/L of Kanamycin and 50 mg/L of Rifampicin, and cultured at 28° C. and 220 rpm with shaking to an OD_{600 nm} value of 0.8-1.0, to obtain Bacillus infection solution.

III. Obtaining the to Generation of Primary Transgenic Plants

1. Taking complete watermelon seeds, carefully peeling off the seed coat (to avoid hurting the seed kernels); firstly disinfecting with 10% (m/v) sodium hypochlorite aqueous solution for 15 min, then washing it with sterile water 3 times, and gently placing in a Petri dish containing BM medium (autoclaved), and culturing under dark at 28° C. for 3 days.

2. After step 1 is completed, taking healthy and sprouting seed kernels, slicing from the paraxial proximal end (size 1.5 mm×1.5 mm) to obtain explants, and placing the explants in a Petri dish (size 9 cm) containing 10 mL of MS liquid medium.

3. Taking out the Petri dish, adding 50 μL of Agrobacterium infection solution into it, and soaking for 10 min.

4. After step 3 is completed, taking out the Petri dish, discarding the bacterial solution, blotting the excess bacterial solution with sterile filter paper, and then placing it on a co-culture medium and co-culturing under dark at 28° C. for 4 days.

5. After step 4 is completed, transferring the explants to selective medium 1, and cultured at 25° C. with alternate light and dark (14 h light/10 h dark; light intensity is about 2000 lx) for 2-4 weeks (subcultured once a week).

6. After step 5 is completed, transferring the explants to selective medium 2, and cultured at 25° C. with alternate light and dark (14 h light/10 h dark; light intensity is about 2000 lx) for 2-4 weeks (subcultured once a week) to obtain green buds.

7. After step 6 is completed, the green buds are transferred to a bud elongation medium, and cultured at 25° C. with alternate light and dark (14 h light/10 h dark; light intensity is about 2000 lx) for 4 weeks to obtain resistant seedlings. During the period, subculture is performed once a week.

8. After step 7 is completed, transferring the resistant seedlings to the rooting medium, and cultured at 25° C. with alternate light and dark (14 h light/10 h dark; light intensity is about 2000 lx) for 7 days to obtain regenerated plants, that is, the T₀ generation of primary transgenic plants.

IV. Identification of T₀ Generation Primary Transgenic Plants

1. Molecular Identification

Using the genomic DNA from the leaves of the T₀ generation of primary transgenic plants obtained in step III as a template, BE3-IDF: 5′-CATACCTCCCAGAACACAAATAAGC-3′ (sequence 10 in the sequence listing) and BE3-IDR: 5′-ACTGAAGGGCAATAGTGAAGAATGT-3′ (sequence 11 in the sequence listing) as primers to perform PCR, so as to obtain PCR amplification products. Agarose gel electrophoresis was performed on the PCR amplified products. T₀ generation of primary transgenic plants with a target band of about 500 bp are T₀ generation of positive transgenic plants.

The PCR is performed with the method as above, except that the genomic DNA of the leaves of the T₀ generation of primary transgenic plant were substituted with water, the recombinant plasmid pBSE901-ALS, and the genomic DNA of the leaves of non-transgenic watermelon plants, respectively.

The results showed that there was no target band after PCR amplification using water and genomic DNA of leaves of non-transgenic watermelon plants as templates; while PCR amplification using the recombinant plasmid pBSE901-ALS had a target band of about 500 bp.

2. Bar Immunoassay Test Paper Identification

(1) Processing of Test Samples

Taking 0.1 g of the leaves of the T₀ generation of primary transgenic plants obtained in step III, putting it into a 2 mL of centrifuge tube, and grinding it with distilled water to obtain a sample solution.

(2) Sample Detection

After step (1) is completed, inserting the Bar immunoassay test strip (a product of Beijing Ao Chuang Jin Biao Biotechnology Co., Ltd.) vertically into the centrifuge tube, and the test strip end will be submerged into the sample solution to a depth of about 0.5 cm. Taking it off and reading the test results after 1 min.

(3) Determination of Results

The detection line and control line may generally appear within 1-2 minutes. The detection standard is: only one purple-red quality control line appearing on the test strip means a negative result; two purple-red bands appearing on the detection strip (one is purple-red detection line, and the other is purple-red quality control line) means a positive result.

T₀ generation of primary transgenic plants with leaves that can obtain two purplish red bands are T₀ generation positive transgenic plants.

A total of 199 T₀ positive transgenic plants were identified.

V. Molecular Detection of Mutation Types

1. Using genomic DNA of leaves of T₀ positive transgenic plants as templates, and ALS-190-IDF: 5′-CGTCACCAATGTCTTCGCTTA-3′ (sequence 12 in the sequence listing) and ALS-190-IDR: 5′-CAGGCTTCTTAGATTCAGATACCA-3′ (sequence 13 in the sequence listing) as primers to perform PCR amplification to obtain PCR amplification products and sequence.

Sequencing results showed that among the 199 T₀ positive transgenic plants, the genotypes of 154 strains were completely consistent with those of the wild type (i.e., non-transgenic watermelon), the target regions were not edited. All or part of the target regions C is mutated to T and all are heterozygous mutations in 45 strains, with a mutation rate of 22.61%. There are two types of mutations in the CLALS gene in heterozygous mutant stains: one is mutant gene 1 (shown in sequence 3 in the sequence listing), which is obtained by mutating C at position 568 from the 5′ end of sequence 1 (i.e., CLALS gene) in the sequence listing to T; the other is mutant gene 2 (shown in sequence 5 in the sequence listing), which is obtained by mutating C at positions 568 and 569 from the 5′ end of sequence 1 (i.e., CLALS gene) in the sequence listing to T. Mutant gene 1 encodes mutant protein 1 shown in sequence 4 in the sequence listing, and mutant gene 2 encodes mutant protein 2 shown in sequence 6 in sequence listing. Compared with the CLALS protein, in mutant protein 1, proline at 190 is mutated to serine, and in mutant protein 2, proline at position 190 is mutated to leucine.

The heterozygous mutant strain with the mutant gene 1 was named as P190L mutant heterozygote. The heterozygous mutant strain with the mutant gene 2 was named as P190S mutant heterozygote.

VI. Obtaining P190L Homozygous Mutant Strain, P190S Homozygous Mutant Strain, P190L Heterozygous Mutant Strain and P190S Heterozygous Mutant Strain

1. Hybridizing the plant containing the P190L heterozygous mutant strain (as the parent) and the non-transgenic watermelon plant (as the parent) to obtain a crossbred.

2. After step 1 is completed, planting the homozygous seeds to obtain plants.

Plants were identified to determine whether they are transgenic and contain P190L mutation. Plants that are non-transgenic and contain P190L mutation account for about 25%.

3. After step 2 is completed, plants that are non-transgenic and contain P190L mutation are self-crossed, to harvest seeds. The seeds were planted to obtain plants, and the genotype of plants was analyzed.

Plants that are non-transgenic and contain P190L homozygous mutation (i.e., P190L homozygous mutants) account for about 25%.

4. After step 3 is completed, the P190L homozygous mutant strains are self-crossed to culture a large number of offspring with the P190L homozygous mutation.

5. After step 4 is completed, the P190L homozygous mutant strains and the non-transgenic watermelon plants are crossed, and the crossed seeds are the P190L heterozygote mutant strains.

The above steps are repeated, except that “plants containing P190L homozygous mutation” were substituted with “plants containing P190S homozygous mutation” to obtain P190S homozygous mutant strains and P190S homozygous mutant strains.

VII. Identification of the Resistance to the Herbicide Tribenuron

The watermelon seeds to be tested are seeds of non-transgenic watermelon, seeds of P190L homozygous mutant strains, seeds of P190S homozygous mutant strains, seeds of P190L heterozygote mutant strains, or seeds of P190S heterozygote mutant strains.

The experiment was repeated three times, and the steps for each were as follows:

1. Planting 20 watermelon seeds to be tested in the field and culturing routinely to obtain watermelon seedlings to be tested in a two-leaf one-heart period.

2. After step 1 is completed, taking the watermelon seedlings to be tested, spraying the leaves with tribenuron (the spraying dose is 17 g ai/ha; g represents gram, ai represents the active ingredient, and ha represents hectare), and then culturing routinely for 7 days to observe the growth status of the watermelon seedlings to be tested.

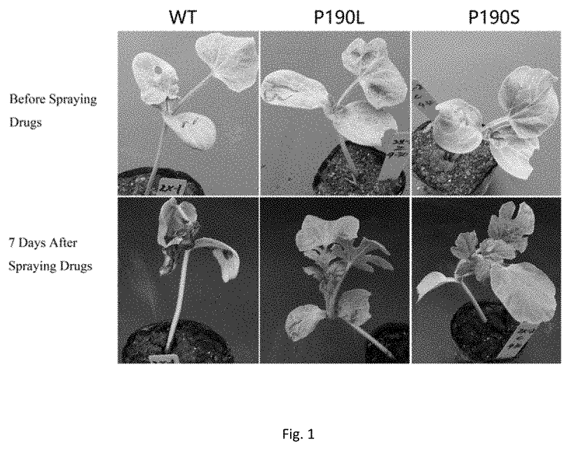

Some experimental results are shown in FIG. 1 (WT represents the seed of non-transgenic watermelon, P190L represents the seed of the P190L homozygous mutant strain, and P190S represents the seed of the P190S homozygous mutant strain). The results showed that the seedlings of seeds of non-transgenic watermelon died quickly after spraying tribenuron (3-7 days after spraying tribenuron), seedlings of seeds of P190L heterozygous mutant strain, seedlings of the seeds of P190S heterozygous mutant strain, seedlings of the P190L homozygous mutant strain and seedlings of the P190S homozygous mutant strain all grew normally. Further, seedlings of the seeds of the P190L homozygous mutant strain and seedlings of the seeds of the P190S homozygous mutant strain grew better than seedlings of the seeds of the P190L heterozygous mutant strain and seedlings of the seeds of P190S heterozygous mutant strain.

The above results indicate that watermelon containing mutant gene 1 and/or mutant gene 2 has obvious resistance to tribenuron.

VIII. Identification of Herbicide Resistance Spectrum

ALS inhibitor herbicides can be divided into five categories according to their chemical structures: 1) Sulfonylurea herbicides, such as mesosulfuron, tribenuron, halosulfuron, bensulfuron, pyrazosulfuron, nicosulfuron; 2) imidazolinones, such as Imazapic; 3) triazopyrimidine herbicides, such as penoxsulam, pyroxsulam, florasulam, and flumetsulam, and the like; 4) pyrimidine salicylic acid herbicides, such as bispyribac; 5) triazolinone herbicides, such as flucarbazone.

Watermelon seeds to be tested are seeds of non-transgenic watermelon, seeds of P190L homozygous mutant strain, seeds of P190S homozygous mutant strain, seeds of P190L heterozygous mutant strain, or seeds of P190S heterozygous mutant strain.

The experiment was repeated three times, and the steps for each are as follows:

1. Planting 5 watermelon seeds to be tested in the field and culturing routinely for 10 days until the cotyledons are flattened to obtain the watermelon seedlings to be tested.

2. After step 1 is completed, taking the watermelon seedlings to be tested and spraying the leaves with tribenuron, halosulfuron, bensulfuron, pyrazosulfuron, flumetsulam, and flucarbazone, respectively (spraying dose of tribenuron, halosulfuron, bensulfuron, pyrazosulfuron, flumetsulam, and flucarbazone were 15, 33.75, 22.5, 24, 48, and 31.5 g ai/ha; g represents gram, ai represents the active ingredient, ha represents hectare), and then culturing for 7 days to observe the growth status of the watermelon seedling to be tested.

3. After step 1 is completed, taking the watermelon seedlings to be tested, spraying the leaves with the same volume of water as the herbicide in step 2, and then culturing for 7 days, to observe the growth status of the watermelon seedlings as a control.

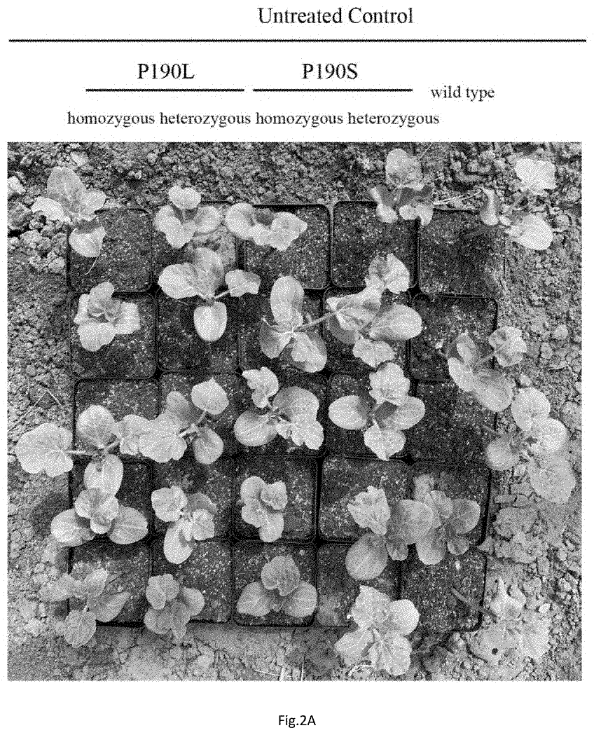

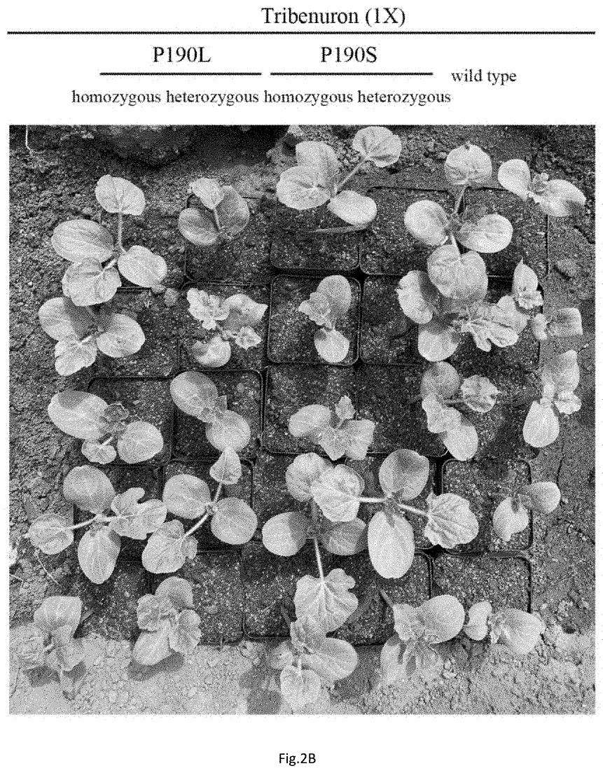

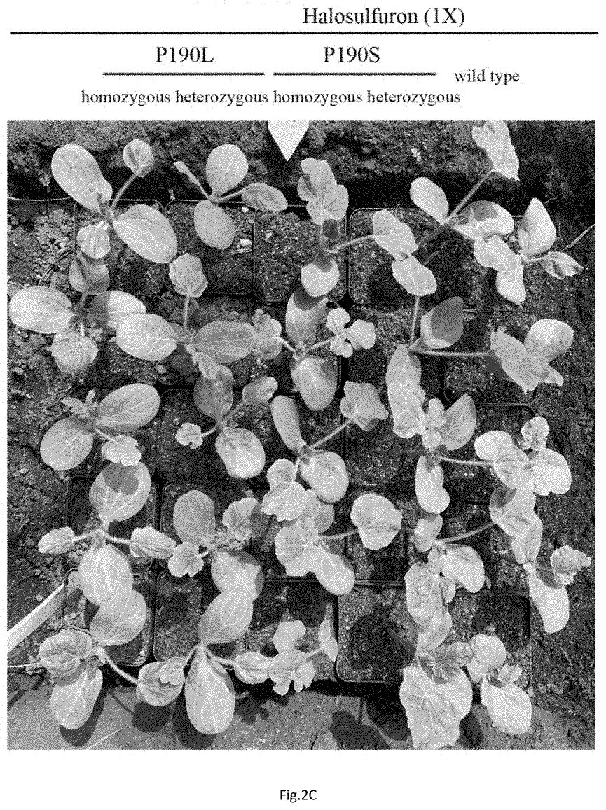

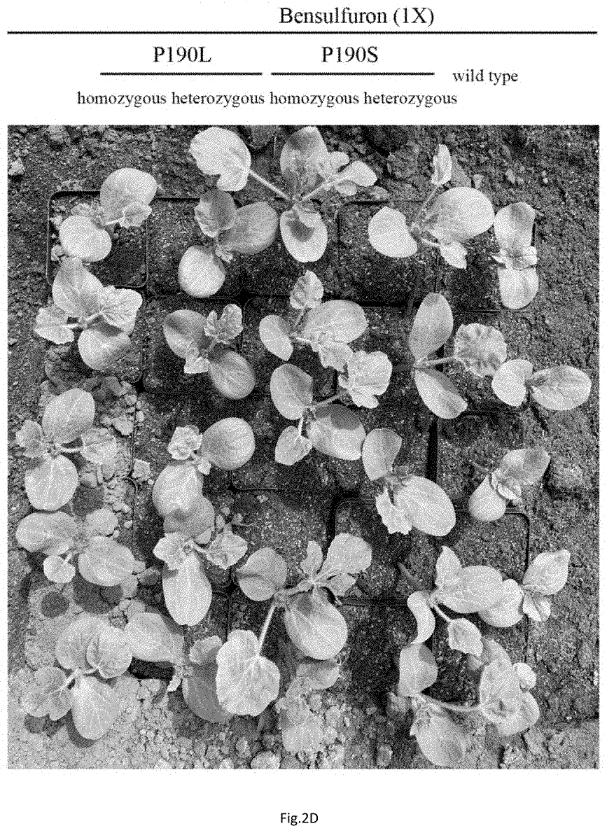

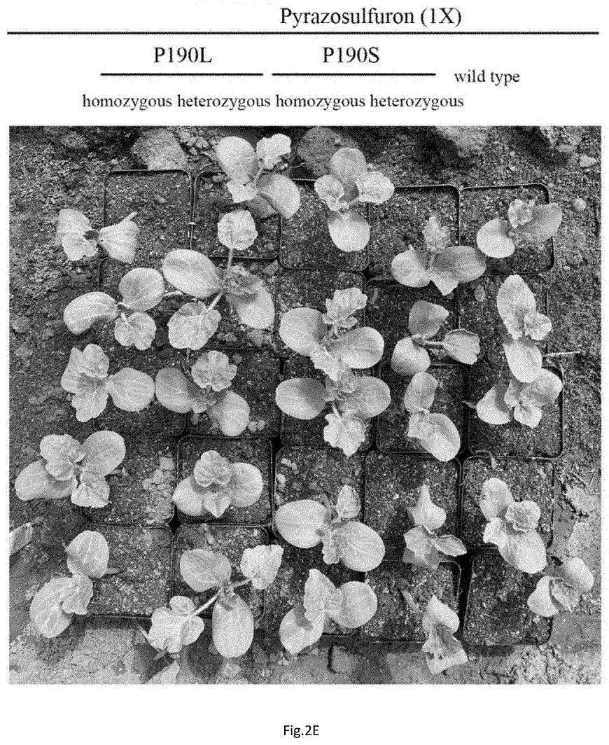

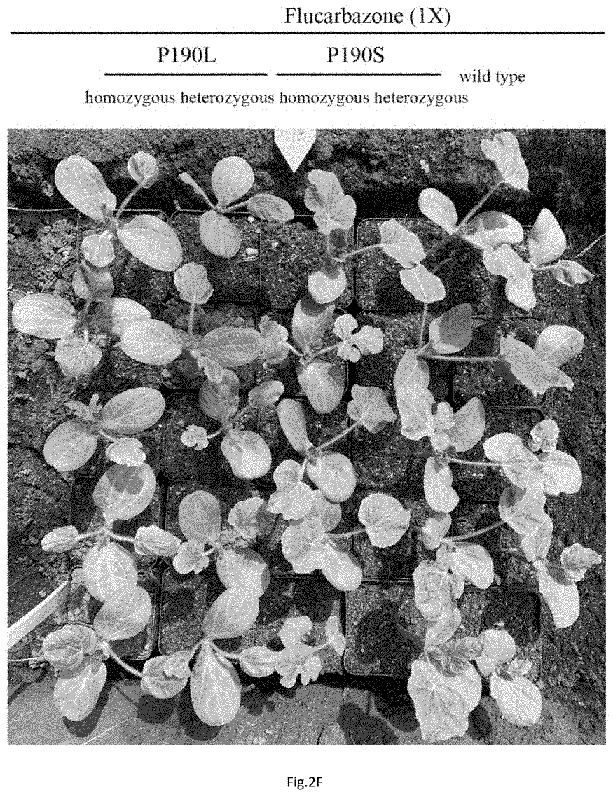

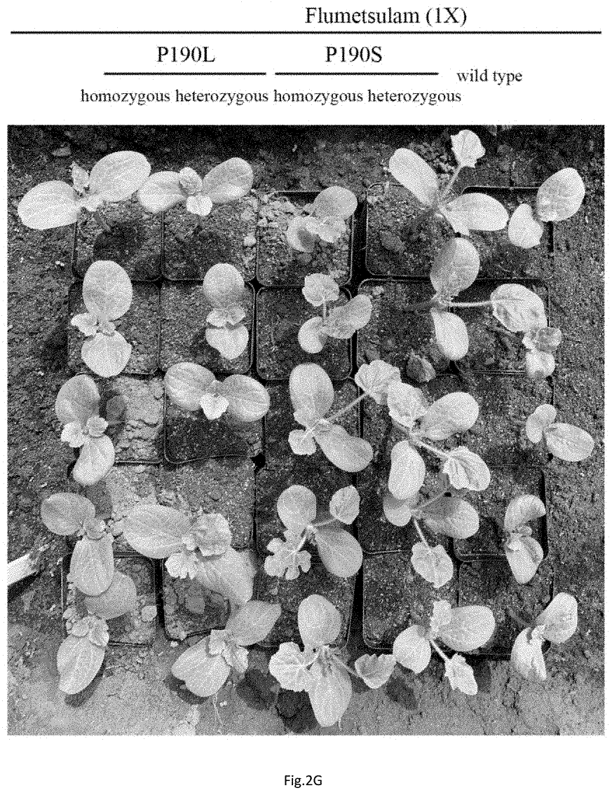

The experimental results are shown in FIG. 2 (the wild type represents non-transgenic watermelon, FIG. 2A represents the control, FIG. 2B represents the watermelon seedlings sprayed with tribenuron, FIG. 2C represents the watermelon seedlings sprayed with halosulfuron, and FIG. 2D represents the watermelon seedlings sprayed with bensulfuron, FIG. 2E represents watermelon seedlings sprayed with pyrazosulfuron, FIG. 2F represents watermelon seedlings sprayed with flucarbazone, and FIG. 2G represents watermelon seedlings sprayed with flumetsulam). The results showed that compared with non-transgenic watermelon, all of the P190L homozygous mutant strain, P190S homozygous mutant strain, P190L heterozygous mutant strain and P190S heterozygous mutant strain show obvious resistance to tribenuron, halosulfuron, bensulfuron, pyrazosulfuron, flucarbazone, and flumetsulam.

It can be seen that P190L homozygous mutant strain, P190S homozygous mutant strain, P190L heterozygous mutant strain and P190S heterozygous mutant strain have broad spectrum resistance to ALS inhibitor herbicides.

### INDUSTRIAL APPLICATION

In the present invention, a P190L heterozygous mutant and a P190S heterozygous mutant are obtained via a plant single base editing system nCas9-PBE, and a P190L homozygous mutant strain (the type of the amino acid residue at position 190 from the N-terminus of the CLALS protein is only a leucine residue), P190S homozygous mutant strain (the type of the amino acid residue at position 190 from the N-terminus of the CLALS protein is only a serine residue), P190L heterozygous mutant strain (the type of the amino acid residue at position 190 from the N-terminus of the CLALS protein is a proline and a leucine residue) and P190S homozygous mutant strain (the type of the amino acid residue at position 190 from the N-terminus of the CLALS protein is a proline residue and a serine residue). Spraying seedlings of the above mutant strains and seedlings of non-transgenic watermelon (the type of the amino acid residue at position 190 from the N-terminus of the CLALS protein is only a proline residue) with tribenuron, and the results showed that the seedlings of non-transgenic watermelon die quickly (3-7 days after spraying tribenuron), all of the seedlings of the P190L heterozygous mutant strain, the seedlings of the P190S heterozygous mutant strain, the seedlings of the P190L homozygous mutant strain, and the seedlings of the P190S homozygous mutant stain grew normally. Moreover, the seedlings of the P190L homozygous mutant strain and the seedlings of the P190S homozygous mutant strain have a better growth status than that of the seedlings of the P190L heterozygous mutant strain and the P190S heterozygous mutant strain. It can be seen that the type of the amino acid residue at position 190 of CLALS protein from the N-terminus can be used as a detection target to predict the herbicide resistance of a watermelon to be tested. The present invention has a great application value.

What is claimed is:

1. A plant or a part of plant, wherein the plant is modified to comprise a polynucleotide encoding an acetolactate synthase (ALS) comprising a non-proline residue at a position corresponding to position 190 of SEQ ID NO: 2.

2. The plant or a part of the plant of claim 1, wherein the plant is modified to comprise an exogenous polynucleotide encoding an ALS comprising a non-proline residue at a position corresponding to position 190 of SEQ ID NO: 2.

3. The plant or a part of the plant of claim 1, wherein the plant is modified to comprise a mutation in the endogenous polynucleotide encoding ALS, wherein the resulting mutated polynucleotide encodes an ALS comprising a non-proline residue at a position corresponding to position 190 of SEQ ID NO: 2.

4. The plant or a part of the plant of claim 1, wherein the the non-proline residue is a serine residue or a leucine residue.

5. The plant or a part of the plant of claim 1, wherein the plant is watermelon.

6. The plant or a part of the plant of claim 1, wherein the modification result in increased herbicide resistance of plant.

7. The plant or a part of the plant of claim 6, wherein the herbicide resistance is ALS inhibitor herbicides.

8. The plant or a part of the plant of claim 6, wherein the herbicide is Y1) or Y2) or Y3) or Y4) or Y5): Y1) sulfonylurea herbicides; Y2) triazopyrimidine herbicides; Y3) triazolinone herbicides; Y4) pyrimidine salicylic acid herbicides; Y5) imidazolinones.

9. The plant or a part of the plant of claim 8, wherein the sulfonylurea herbicides are tribenuron, halosulfuron, bensulfuron, nicosulfuron, mesosulfuron, thiensulfuron, or rimsulfuron;
the triazopyrimidine herbicides are flumetsulam, penoxsulam, pyroxsulam, or florasulam;
the triazolinone herbicide is flucarbazone;
the pyrimidine salicylic acid herbicide is bispyribac;
the imidazolinone is Imazapic.

10. The plant or a part of the plant of claim 1, wherein the part of plant comprise plant cell or plant tissue or plant organs, and the plant organs comprise seed, leaf, flower, fruit, stem or root.

11. The plant or a part of the plant of claim 1, wherein the amino sequence of acetolactate synthase (ALS) comprising a non-proline residue at a position corresponding to position 190 of SEQ ID NO: 2 comprise SEQ ID NO: 4 or SEQ ID NO: 6.

12. The plant or a part of the plant of claim 1, wherein the polynucleotide encoding an acetolactate synthase (ALS) comprising a non-proline residue at a position corresponding to position 190 of SEQ ID NO: 2 has a nucleotide substitution of C to T at a position corresponding to position 568 of SEQ ID NO: 1 or has a nucleotide substitution of C to T at a position corresponding to position 568 and 569 of SEQ ID NO: 1.

13. The plant or a part of the plant of claim 1, wherein the nucleotide sequence of the polynucleotide encoding an acetolactate synthase (ALS) comprising a non-proline residue at a position corresponding to position 190 of SEQ ID NO: 2 comprise SEQ ID NO: 3 or SEQ ID NO: 5.

14. A method of producing the plant of claim 1, comprising
crossing the the plant of claim 1 with wild type plant;
optionally performing one or more rounds of selfing and/or crossing;
and optionally selecting after each round of selfing and/or crossing for a plant that comprises said increased herbicide resistance.

15. A CLALS protein is c2) or c3) as follows:
c2) a protein with the amino acid sequence shown in sequence 4 in the sequence listing;
c3) a protein with the amino acid sequence shown in sequence 6 in the sequence listing;

16. A nucleic acid molecule encoding the CLALS protein of claim 15.

17. The nucleic acid molecule according to claim 16, wherein the nucleic acid molecule is a DNA molecule shown in d2) or d3) as follows:
d2) a DNA molecule with a nucleotide sequence shown in sequence 3 in the sequence listing;
d3) a DNA molecule with a nucleotide sequence shown in sequence 5 in the sequence listing.

18. A method for predicting the herbicide resistance of watermelon to be tested, which is S1) or S2) or S3):
S1) detecting the type of the amino acid residue at position 190 from the N-terminus of the CLALS protein according to claim 15 of the watermelon to be tested;
the herbicide resistance of a watermelon to be tested in which “the type of the amino acid residue at position 190 from the N-terminus of the CLALS protein is only a non-proline residue” or a watermelon to be tested in which “the type of the amino acid residue at position 190 from the N-terminus of the CLALS protein is a non-proline residue and a proline residue” is stronger than that of a watermelon to be tested in which “the type of the amino acid residue at position 190 from the N-terminus of the CLALS protein is only a proline residue”;
S2) detecting the nucleotide sequence of the 190^{th} codon in the specific transcript of the total RNA of a watermelon to be tested; the specific transcript is the RNA transcribed from the gene encoding the CLALS protein according to claim 15, and the first codon is the start codon;
the herbicide resistance of a watermelon to be tested in which “the nucleotide sequence of the 190^{th} codon in a specific transcript only encodes a non-proline” or a watermelon to be tested in which “the nucleotide sequence of the 190^{th} codon in a specific transcript only encodes a non-proline and a proline” is stronger than that of a watermelon to be tested in which “the nucleotide sequence of the 190^{th} codon in a specific transcript only encodes a proline”;
S3) detecting the type of the nucleotide at positions 568 and 569 from 5′ end of the gene encoding the CLALS protein according to claim 15 in the total DNA of a watermelon to be tested;
the herbicide resistance of a watermelon to be tested in which “the type of the nucleotides at positions 568 and 569 from 5′ end of the gene encoding the CLALS protein is only c” is weaker than F1 or F2 or F3; F1 is a watermelon to be tested in which the type of the nucleotides at both positions 568 and 569 from 5′ end of the gene encoding the CLALS protein does not include c; F2 is a watermelon to be tested in which the type of the nucleotides at position 569 from 5′ end of the gene encoding the CLALS protein includes c, and the type of the nucleotides at position 568 does not include c; F3 is a watermelon to be tested in which the type of the nucleotides at position 568 from 5′ end of the gene encoding the CLALS protein includes c, and the type of the nucleotides at position 569 does not include c.

19. The method according to claim 18, wherein:
the amino acid type of the non-proline residue in “the type of the amino acid residue at position 190 from the N-terminus of the CLALS protein is only a non-proline residue” may be one or two;
the amino acid type of the non-proline in “the nucleotide sequence of the 190^{th} codon in a specific transcript only encodes a non-proline” may be one or two.

20. The method according to claim 18, wherein the herbicide targeting the CLALS protein is Y1) or Y2) or Y3) or Y4) or Y5): Y1) sulfonylurea herbicides; Y2) triazopyrimidine herbicides; Y3) triazolinone herbicides; Y4) pyrimidine salicylic acid herbicides; Y5) imidazolinones;
wherein the sulfonylurea herbicides are tribenuron, halosulfuron, bensulfuron, nicosulfuron, mesosulfuron, thiensulfuron, or rimsulfuron;
the triazopyrimidine herbicides are flumetsulam, penoxsulam, pyroxsulam, or florasulam;
the triazolinone herbicide is flucarbazone;
the pyrimidine salicylic acid herbicide is bispyribac;
the imidazolinone is Imazapic.
